(19) United States
(12) Patent Application Publication
(10) Pub. No.: US 2024/0402106 A1
(43) Pub. Date: Dec. 5, 2024

(54) SENSOR DEVICE FOR MEASURING A GAS CONCENTRATION, AND METHOD FOR DETERMINING A GAS CONCENTRATION

(71) Applicant: Infineon Technologies AG, Neubiberg (DE)

(72) Inventors: Christoph STEINER, St. Margarethen a. d. Raab (AT); Gerald HANSEKOWITSCH, St. Veit am Vogau (AT)

(21) Appl. No.: 18/672,875

(22) Filed: May 23, 2024

(30) Foreign Application Priority Data
Jun. 2, 2023 (DE) 102023205187.7

(51) Int. Cl. G01N 25/18; G01N 33/00

(52) U.S. Cl. CPC G01N 25/18; G01N 33/0027

(57) ABSTRACT

A sensor device for measuring a gas concentration includes a sensor element a cavity with an opening for receiving a gas, and a resistor element arranged in the cavity, and a power supply unit, which is configured to apply a voltage to the sensor element. The sensor device further includes a control unit, which is configured to set a first voltage of the power supply unit and detect a first output signal of the sensor element at the applied first voltage, and to set a second voltage of the power supply unit and detect a second output signal of the sensor element at the applied second voltage. The second voltage is different from the first voltage. The sensor device further includes an evaluation unit, which is configured to determine the gas concentration based on the first output signal and the second output signal.

### CROSS-REFERENCE TO RELATED APPLICATION

This application claims priority to German Patent Application No. 102023205187.7 filed on Jun. 2, 2023, the content of which is incorporated by reference herein in its entirety.

### TECHNICAL FIELD

The present disclosure relates to a sensor device for measuring a gas concentration and to a method for determining a gas concentration, in particular a concentration of hydrogen.

### BACKGROUND

Gas concentration sensors, e.g., thermal conductivity sensors, can be used for example in the automotive sector or in a wide range of industrial applications. Here, such sensors can provide measured values that indicate a thermal conductivity of an analytical gas. However, these measured values may be tainted with deviations or offset effects that are affected by other properties of the gas to be measured. For example, the measurement of thermal conductivity can be highly dependent on the ambient pressure of the gas to be measured. Inaccurate measured values can pose a significant safety risk, especially in the case of applications in the automotive sector. Manufacturers and designers of gas concentration sensors are therefore constantly striving to improve their products. In particular, it may be desirable to provide thermal conductivity sensors that take into account the offset effects mentioned in order to provide reliable and accurate measurement results. In addition, it may be desirable to provide suitable methods for operating such thermal conductivity sensors.

### SUMMARY

There is a need for a gas sensor that can be used to determine a gas concentration, in particular a hydrogen content, with higher operational reliability.

Based on this, a sensor device according to the main claim is proposed. Advantageous refinements are specified in the subclaims.

A first aspect of the present disclosure relates to a sensor device for measuring a gas concentration, which includes a sensor element with a cavity, an opening of the cavity for receiving a gas, and a resistor element arranged in the cavity. The sensor device further includes a power supply unit, which is configured to apply a voltage to the sensor element, and a control unit. The control unit is configured to set a first voltage of the power supply unit and detect a first output signal of the sensor element at the applied first voltage, and to set a second voltage of the power supply unit and detect a second output signal of the sensor element at the applied second voltage. The second voltage is different here from the first voltage. The sensor device further includes an evaluation unit, which is configured to determine the gas concentration based on the first output signal and the second output signal.

A second aspect of the present disclosure relates to a method for determining a gas concentration. The method includes surrounding a resistor element of a sensor element with a gas, applying a first voltage to the sensor element, detecting a first output signal of the sensor element with the first voltage applied, applying a second voltage to the sensor element, which is different from the first voltage, detecting a second output signal of the sensor element with the second voltage applied, and determining the gas concentration based on the first output signal and the second output signal.

A person skilled in the art will recognize further features and advantages of the disclosure when reading the detailed description that follows and when looking at the accompanying drawings.

### BRIEF DESCRIPTION OF THE DRAWINGS

The present disclosure is described by way of illustration and in nonlimiting fashion in the figures of the accompanying drawings, in which identical reference numerals refer to similar or identical elements. The elements in the drawings are not necessarily depicted to scale with respect to one another. The features of the various examples shown can be combined, unless they are mutually exclusive.

FIG. 1 shows a first example implementation of a sensor device 100 for measuring a gas concentration.

FIG. 2 shows a cross section of an example sensor element 101 for use in a sensor device 100 according to the disclosure.

FIG. 3 shows a cross section of another example sensor element 101 for use in a sensor device 100 according to the disclosure.

FIG. 4 shows a circuit diagram of an example bridge circuit 400 for use in a sensor device 100 according to the disclosure.

FIG. 5 shows a graph for illustrating the typically significant dependence of the measured values on the pressure of the surrounding gas in the cavity 102 of a thermal conductivity sensor when a high supply voltage V₂ is applied.

FIG. 6 shows an example timing diagram for the supply voltage Vᵢ of a sensor element 100 according to the disclosure.

FIG. 7 shows a graph for illustrating the approximate linear dependence of the measured values on the pressure of the surrounding gas in the cavity 102 of a typical thermal conductivity sensor element when a low first supply voltage V₁ is applied.

FIG. 8 shows a flowchart of a method for operating a thermal conductivity sensor according to the disclosure.

### DETAILED DESCRIPTION

The implementations described below give a detailed description of thermal conductivity sensors (or thermal conductivity gas sensors) and methods for operating such sensors according to this disclosure. Thermal conductivity sensors, as described here, can be used in particular as hydrogen sensors for the detection of hydrogen and/or hydrogen concentrations. Hydrogen sensors can be used in a variety of applications, such as in the automotive sector or in industrial applications. For example, hydrogen sensors can be used to detect hydrogen exhaust gases, to monitor exhaust gases, to monitor batteries, to detect hydrogen leaks, to detect hydrogen in industrial plants, etc.

With regard to the climate goals, the automotive industry is promoting and developing the production of hydrogen-powered vehicles. Fuel cell cars can be seen as a breakthrough for electromobility and can contribute significantly to reducing the CO2 emissions. The thermal conductivity sensors described here improve the hydrogen technology and can thus at least partially contribute to achieving the set climate goals. The thermal conductivity sensors described here provide a simple and efficient way to compensate for offset effects. By comparison, the manufacture and design of traditional sensors can be more complex and require a higher number of components, resulting in increased resource consumption. The thermal conductivity sensors described here save resources and can contribute to energy savings. Overall, improved thermal conductivity sensors according to the disclosure and methods for operating such sensors can contribute to green technology and green energy solutions, e.g., to climate-friendly solutions with reduced energy consumption.

FIG. 1 shows a first example implementation of a sensor device 100 for measuring a gas concentration. The sensor device 100 comprises a sensor element 101, which can be a thermal conductivity sensor. The sensor element 101 comprises a resistor element 104, which can be in the form of a free-floating beam or wire. The resistor element 104 can be manufactured, for example, based on MEMS technology (micro-electromechanical systems). The resistor element 104 is arranged in a cavity 102 in which it is exposed to the gas to be measured. In other words, an analytical gas can surround the resistor element 104 in the cavity 102. The gas to be measured can be introduced into the cavity 102 using an opening 103 (not shown in the figure for reasons of clarity). When a supply voltage Vᵢ is applied to the resistor element 104 as illustrated, the resistor element 104 can act as a heating wire and heat up to a stable characteristic temperature Tᵢ above the ambient temperature. The resistor element 104 can release thermal energy to the surrounding gas, which is illustrated in FIG. 1 by small arrows pointing away from the resistor element 104. When the stable characteristic temperature Tᵢ is reached, the total heat loss from the resistor element 104 may correspond to the energy produced by the supply voltage Vᵢ. The higher the thermal conductivity of the gas in the cavity 102, the greater its cooling effect and the lower the temperature of the resistor element 104 at a constant supply voltage Vᵢ. In one example, the resistor element 104 can be a PTC (positive temperature coefficient) resistor configured to conduct electrical currents better at low temperatures than at high temperatures. This means that the resistance value of the resistor element 104 can be lower at low temperatures than at high temperatures. As a result, the electrical current through the resistor element 104 can be a measure of the thermal conductivity of the surrounding gas.

The sensor device 100 in this example further comprises a power supply 110 configured to apply the supply voltage Vᵢ to the resistor element 104. The power supply 110 can output different supply voltages Vᵢ, in particular a first supply voltage V₁ and a second supply voltage V₂ different therefrom. The sensor device 100 further comprises a control unit 120, which is electronically coupled to the power supply 110. The control unit 120, for example a control circuit, may be configured to supply the power supply 110 with a trigger signal, based on which the power supply 110 applies a supply voltage Vᵢ to the resistor element 104. For example, the control unit 120 may be configured to output a first and a second trigger signal in order to trigger application of the first supply voltage V₁ and the second supply voltage V₂, respectively. The second supply voltage V₂ may be greater than the first supply voltage V₁, so that the associated stable characteristic second temperature T₂ of the resistor element 104 at the second supply voltage V₂ is higher than the stable characteristic first temperature T₁, or vice versa. The control unit 120 is also configured to acquire a sensor value from the sensor element 101. For example, the control unit 120 acquires a first output value O₁ of the sensor element 101 after a predefined period t₁ with the first supply voltage V₁ applied and a second output value O₂ after a further predefined period t₂ with the second supply voltage V₂ applied. The predefined periods t₁, t₂ may be defined in such a way that a constant temperature, e.g., the stable characteristic first temperature T₁, T₂, has arisen after the respective supply voltage V₁, V₂ has been applied. The first and second output signals O₁, O₂ can be an electrical current through the resistor element 104 when the characteristic stable first and second temperatures T₁, T₂ of the resistor element 104 have arisen.

The sensor device 100 further comprises an evaluation unit 130, for example an evaluation circuit, which is electronically coupled to the control unit 120 and is configured to receive the first output signal O₁ and the second output signal O₂ from the control unit 120 and to determine a concentration of a gas in the cavity 102 of the sensor element 101 therefrom. For example, the evaluation unit 130 is configured to compensate the first output signal O₁ or a signal formed based on the first output signal O₁ using the second output signal O₂ or a further signal formed based on the second output signal O₂, or vice versa. For example, the second output signal O₂ corresponds to a sensor measured value at a high applied supply voltage V₂, e.g., 5 V, that is to say a high temperature T₂ of the resistor element 104, and depends primarily on the thermal conductivity of the surrounding gas, while the first output signal O₁ corresponds to a sensor measured value at a low applied supply voltage V₁, e.g., 1 V, that is to say a lower temperature T₁ of the resistor element 104, and depends primarily on the ambient pressure of the analytical gas in the cavity 102. This is due to the fact that the gas pressure surrounding the resistor element 104 produces a mechanical tension on the surfaces thereof, which in turn is reflected in a mechanical tension on the piezo resistors, ultimately leading to a pressure-dependent measurement signal. Thus, the evaluation unit 130 may be configured to determine the gas pressure from the first output signal O₁, and to correct or compensate the second output signal O₂ using the determined gas pressure, for example using a calibration table. The evaluation unit 130 may also be configured to output the corrected output signal or to determine the gas concentration from the corrected first output signal and to output a measurement signal based on the determined gas concentration.

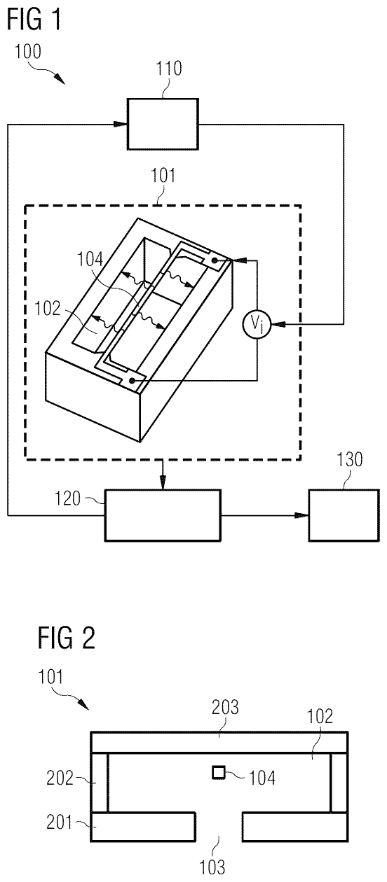

FIG. 2 shows a cross section of an example sensor element 101. The sensor element 101 may be a semiconductor component comprising a substrate 201, side wall elements 202 and a cover 203, which together form a boundary of the cavity 102 and thus define the cavity. In this example, the substrate 103 comprises the opening 103 to the surroundings of the sensor element 101, so that analytical gas to be measured can enter or be introduced into the cavity 102. The opening 103 can alternatively also be arranged in a side wall element 202 or in the cover 203. The resistor element 104 is arranged in the cavity 102 so that it can come into contact with the gas in the cavity. For example, the resistor element 104 is a MEMS wire that forms a piezoresistive element to which the supply voltage Vᵢ can be applied as shown in FIG. 1.

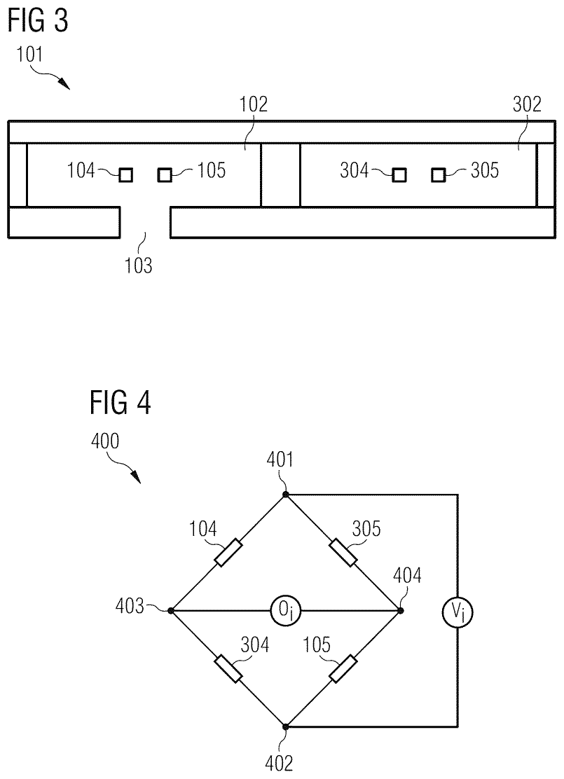

FIG. 3 shows a cross section of another example sensor element 101. Compared with the sensor element 101 of FIG. 2, this sensor element 101 further comprises another resistor element 105, which is also arranged in the cavity 102, a sealed reference cavity 302 without an opening to the surroundings of the sensor element 101, and two reference resistor elements 304, 305, which are arranged in the reference cavity 302. The reference cavity may be filled with a reference gas, such as air, oxygen or nitrogen. The supply voltage Vᵢ can be applied to the resistor elements 104, 105 and to the reference resistor elements 304, 305, which can e.g., all be connected in parallel with one another. Output signals of the reference resistor elements 304, 305 can be used by the evaluation unit 130 to correct or compensate output signals of the resistor elements 104, 105, for example using a calibration table. For example, the gas in the reference cavity 302 has a significantly lesser or greater thermal conductivity than the gas to be measured, for example hydrogen or a gas composition containing hydrogen, that is introduced into the cavity 102. In addition, the evaluation unit 130 can use the gas pressure enclosed in the reference cavity 302 at a given temperature to calibrate or compensate the output signals O₁, O₂.

FIG. 4 shows a circuit diagram of a bridge circuit 400, which a sensor element 101 according to the disclosure may contain. The bridge circuit 400 may correspond to a Wheatstone bridge circuit having two resistor elements 104, 105 and two reference resistor elements 304, 305, according to the sensor element of FIG. 3. The resistors can be interconnected as shown in the circuit diagram. The resistance value of the resistor elements 104, 105 can change depending on the presence and concentration of an analytical gas. The resistance value of the reference resistor elements 304, 305 can remain substantially constant. For example, each of the resistors may be similar to the resistor element 104 in FIG. 1. The supply voltage Vᵢ is applied by the power supply unit 110 between a first node 401 and a second node 402 of the bridge circuit 400.

The bridge circuit 400 may further contain a coupling (not shown) to the control unit 120 for providing an output signal Oᵢ that indicates the thermal conductivity and/or the gas pressure of an analytical gas. In particular, the bridge circuit 400 may be configured so that it measures and outputs a voltage difference between a third node 403 and a fourth node 404 as the output signal Oᵢ, as shown in FIG. 4. The third node 403 may be arranged between the first resistor element 104 and the first reference resistor element 304, while the fourth node 404 may be arranged between the second resistor element 105 and the second reference resistor element 305. Thus, the first resistor element 104 and the first reference resistor element 304 can form a first half-bridge, while the second resistor element 105 and the second reference resistor element 305 form a second half-bridge of the bridge circuit 400.

During operation of the bridge circuit 400, a supply voltage can be applied, as shown in FIG. 4. During a heating phase with the first supply voltage V₁ applied, e.g., 1 V, the bridge circuit 400 can heat up to a characteristic stable first temperature T₁, at which the bridge circuit 400 has become (in particular completely) sensitive to a gas pressure of the analytical gas. Due to differences in the pressure of the reference gas and the pressure of the analytical gas, the bridge circuit 400 can output a non-zero output voltage, which can indicate the gas pressure of the analytical gas, as the first output signal O₁ in this case. During a second heating phase with the second supply voltage V₂ applied, e.g., 5 V, the bridge circuit 400 can heat up to a characteristic stable second temperature T₂, at which the bridge circuit 400 has become (in particular completely) sensitive to a thermal conductivity of the analytical gas. Due to differences in the thermal conductivities of the reference gas and the analytical gas, the bridge circuit 400 can output a non-zero output voltage, which can indicate the thermal conductivity of the analytical gas, as the second output signal O₂ in this case. Based on the output voltages O₁, O₂ provided, the evaluation unit 130 can determine the analytical gas and/or a concentration of the analytical gas.

The bridge circuit 400 may be referred to as a sensor element hereinbelow. It should be noted that the bridge circuit 400 is example and can be replaced by any other half-bridge circuit or bridge circuit configured to provide a measured value indicating the gas pressure or the thermal conductivity of an analytical gas. Accordingly, the sensor elements described here may correspond to or contain a bridge circuit or a half-bridge circuit. The thermal conductivity sensors described here are not limited to the example Wheatstone bridge circuit 400 of FIG. 4.

It should be noted that the thermal conductivity sensors described here may contain further circuit components, such as a switch, a signal amplifier, an analog-to-digital converter, etc. However, these components do not necessarily have to be considered as part of a sensor element 101. The operation of these additional components does not necessarily have to depend on the prevailing temperature. In contrast, the operation of the bridge circuit 400 and the value of the output signal Oᵢ, e.g., the output voltage, may depend on the temperature T₁ of the bridge circuit.

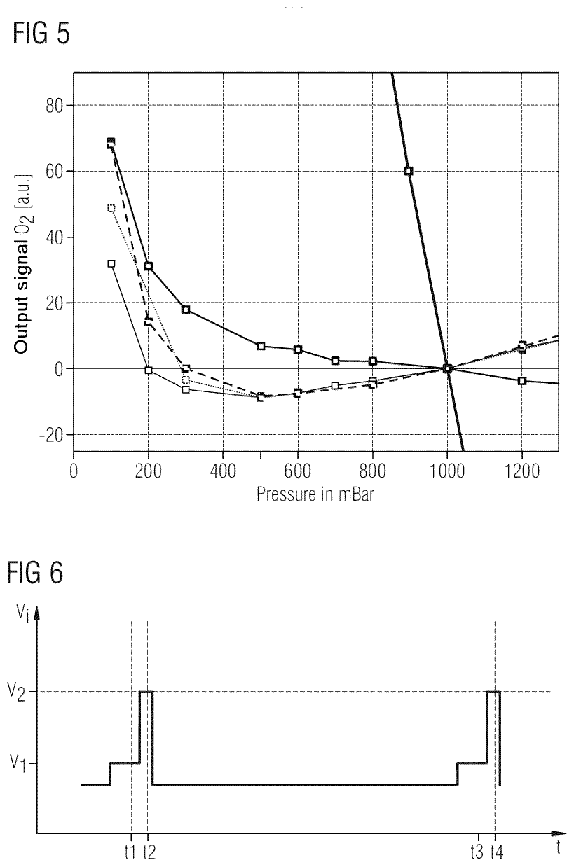

FIG. 5 is a graph for illustrating the typically significant dependence of the measured values on the pressure of the surrounding gas in the cavity 102 of a typical thermal conductivity sensor element when a high supply voltage V₂ (output signal O₂ in any units) is applied. The actual values of the output signals O₂, that is to say e.g., the output voltage of the bridge circuit of FIG. 4, under given conditions can differ from sensor to sensor and can be overlaid with voltage changes caused by an analytical gas. The graph shows a clear dependence of the output signals O₂ on the ambient pressure of the analytical gas. Correction of the second output signal O₂ to reliably determine the thermal conductivity and thus the concentration of the analytical gas is therefore necessary.

FIG. 6 shows an example timing diagram for the supply voltage Vᵢ according to the disclosure. In a first step, a non-zero first supply voltage V₁ is applied to the sensor element 101, for example the bridge circuit 400 from FIG. 4. For example, the first supply voltage V₁ is 1 V. At a defined time t₁ after the first supply voltage V₁ has been applied, in particular after the stable first temperature T₁ of the sensor element 101 has been reached, the control unit 120 detects the first output signal O₁, e.g., the first output voltage of the bridge circuit 400. A second supply voltage V₂, which is higher in this example (e.g., 5 V), is then applied to the sensor element 101. At a defined time t₂ after the second supply voltage V₂ has been applied, in particular after the stable second temperature T₂ of the sensor element 101 has been reached, the control unit 120 detects the second output signal O₂ in an analogous manner. Due to the higher second supply voltage V₂, the second output signal O₂ is also dependent on the thermal conductivity of the gas in the cavity 102, in addition to the pressure dependence mentioned. The first output signal O₁, on the other hand, is primarily dependent on the pressure of the gas in the cavity 102, due to the lower temperature T₁. The control unit 130 may be configured to determine the pressure from the first output signal O₁ in order to calibrate or correct a thermal conductivity of the gas in the cavity 102 that has been determined from the second output signal O₂, e.g., by applying an algorithm to the second output signal O₂ or by looking up in a lookup table. From the corrected conductivity determined, the evaluation unit 130 can determine a concentration of the gas to be measured in the cavity 102 and can output this concentration as a measurement signal for further processing.

A further measurement can be repeated in an analogous manner at a further time t₃ or t₄, for example in order to verify the first measurement and/or to monitor a possible change in the thermal conductivity and thus the gas concentration. A multiplicity of first output signals O₁ can also be determined for every second output signal O₂, for example in order to improve the signal-to-noise ratio and to determine a reliable pressure value. In this case, the evaluation unit 130 is configured to correct the second output signal O₂ with the multiplicity of first output signals O₁ or a signal determined from the multiplicity of first output signals O₁. For example, the evaluation unit 130 determines a mean gas pressure from the multiplicity of first output signals O₁ and corrects the second output signal O₂ or a signal determined from the second output signal O₂, e.g., a thermal conductivity of the analytical gas, using the mean gas pressure, in order to obtain a corrected thermal conductivity. The evaluation unit 130 is further configured to determine the gas concentration, for example a hydrogen concentration, of the analytical gas from the corrected thermal conductivity. If necessary, the evaluation unit 130 may be configured to output an alarm signal if the determined gas concentration falls below and/or exceeds a threshold value.

As an alternative to the timing diagram shown in FIG. 6, the control unit 120 may be configured to apply the first supply voltage V₁ as a higher voltage compared with the second supply voltage V₂. Likewise, the control unit 120 may be configured to apply a third voltage, which may be 0 V, which is different from the first supply voltage V₁ and the second supply voltage V₂, between applying the first supply voltage V₁ and the second supply voltage V₂.

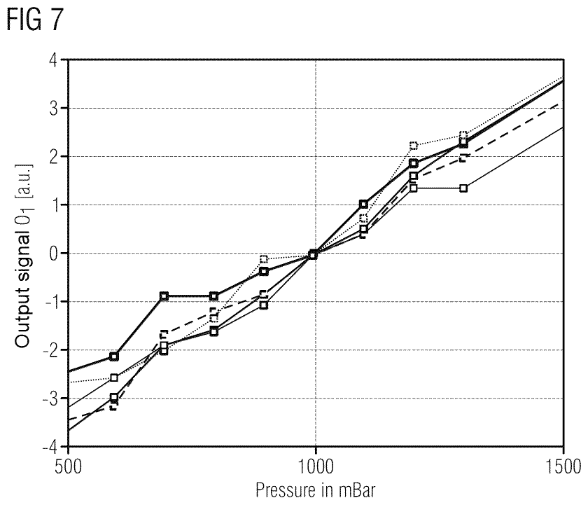

FIG. 7 is a graph for illustrating the approximate linear dependence of the measured values on the pressure of the surrounding gas in the cavity 102 of a typical thermal conductivity sensor element when a low first supply voltage V₁ (output signal O₁ in any units) is applied. The actual values of the output signals O₁, that is to say e.g., the output voltage of the bridge circuit of FIG. 4, under given conditions can differ from sensor to sensor and can be overlaid with voltage changes caused by an analytical gas. The graph shows a clear linear dependence of the output signals O₁ on the ambient pressure of the analytical gas. Thus, the gas pressure of the analytical gas can be reliably determined from the first output signal O₁ and can be used to compensate or correct a determination of the thermal conductivity and thus the concentration of the analytical gas. The sensitivity of thermal conductivity sensors based on piezoresistive wires is approximately 0.03 μV/mbar for a supply voltage V₁ of 1 V. Atmospheric pressure is a very slowly changing process, e.g., any new measurement of the “cold” wire when the first supply voltage V₁ is low can be considered as an update of the present pressure estimate. The evaluation unit 130 can then apply different filter techniques, such as moving average or Kalman filter, to a plurality of first output signals O₁.

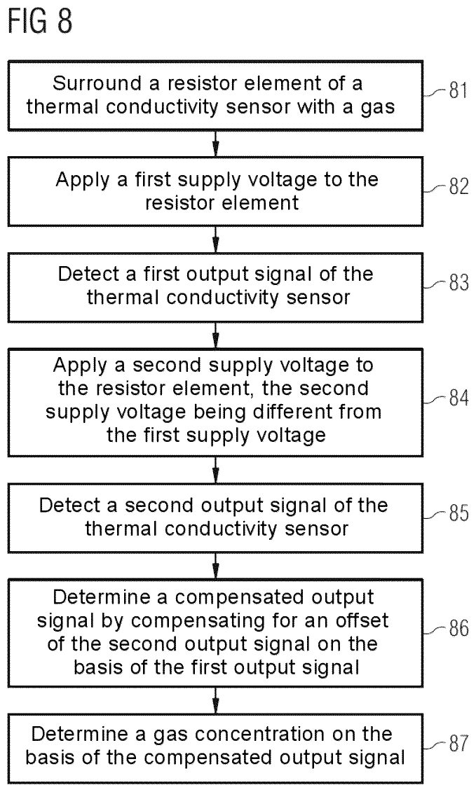

FIG. 8 shows a flowchart of a method for operating a thermal conductivity sensor according to the disclosure. The method is described in a general form in order to qualitatively specify aspects of the disclosure. The method may contain other aspects.

In step 81, a resistor element 104 of a sensor element 101 is surrounded by a gas whose concentration is to be determined. At 82, a first supply voltage V₁ can be applied to the resistor element 104. At 83, the control unit 120 can perform a first measurement at the first supply voltage V₁ for the analytical gas and can detect a first output signal O₁. At 84, a second supply voltage V₂ can be applied to the resistor element 104, the second supply voltage V₂ being able to be higher than the first supply voltage V₁. At 85, the control unit 120 can perform a second measurement at the second supply voltage V₂ for the analytical gas. In this context, a second output signal O₂ can be determined. At 86, the evaluation unit 130 can determine a compensated output signal or measurement signal, e.g., by compensating for an offset of the second output signal O₂ due to an ambient pressure of the analytical gas based on the first output signal O₁. At 87, the evaluation unit 130 can determine the concentration of the analytical gas based on the compensated output signal.

It should be pointed out that the description and the drawings illustrate only the principles of the proposed devices and methods. A person skilled in the art will be able to implement various arrangements which, although not expressly described or shown here, embody, and are included in the extent of, the principles of the implementation. In addition, all examples and implementations outlined in the present document are fundamentally and expressly intended only for explanatory purposes to help the reader understand the principles of the proposed methods and devices. In addition, all statements in this document that describe principles, aspects and implementations of the implementation, and specific examples thereof, are also intended to include their equivalents.

### ASPECTS

Devices and methods according to the disclosure are explained hereinbelow using aspects.

Aspect 1 is a sensor device for measuring a gas concentration, comprising:
a sensor element comprising a cavity with an opening for receiving a gas, and a resistor element arranged in the cavity, a power supply unit, which is configured to apply a voltage to the sensor element, a control unit, which is configured to set a first voltage of the power supply unit and detect a first output signal of the sensor element at the applied first voltage, and to set a second voltage of the power supply unit and detect a second output signal of the sensor element at the applied second voltage, the second voltage being different from the first voltage, and an evaluation unit, which is configured to determine the gas concentration based on the first output signal and the second output signal.

Aspect 2 is a sensor device according to Aspect 1, wherein the first output signal and/or the second output signal depends on the gas concentration and the thermal conductivity of the gas.

Aspect 3 is a sensor device according to one of the preceding aspects, wherein the control unit is configured to reach a first temperature of the resistor element using the first voltage and determine the first output signal at the first temperature, and to reach a second temperature of the resistor element using the second voltage and determine the second output signal at the second temperature, the first temperature being different from the second temperature.

Aspect 4 is a sensor device according to Aspect 3, wherein the first and/or the second temperature are greater than an ambient temperature of the sensor device.

Aspect 5 is a sensor device according to Aspect 3 or 4, wherein the first and/or the second temperature depends on the gas concentration and the thermal conductivity of the gas.

Aspect 6 is a sensor device according to one of the preceding aspects, wherein the control unit is configured to determine a first resistance signal of the sensor element as the first output signal and a second resistance signal of the sensor element as the second output signal.

Aspect 7 is a sensor device according to one of the preceding aspects, wherein the resistor element is in the form of a micro-electromechanical systems structure, in particular in the form of a MEMS wire element.

Aspect 8 is a sensor device according to one of the preceding aspects, wherein the resistor element has piezoresistive properties.

Aspect 9 is a sensor device according to one of the preceding aspects, wherein the sensor element further comprises a sealed reference cavity and a reference resistor element arranged in the reference cavity, and wherein the power supply unit is further configured to apply the voltage to the resistor element and to the reference resistor element of the sensor element.

Aspect 10 is a sensor device according to Aspect 9, wherein the resistor element and the reference resistor element are arranged in a parallel switching configuration.

Aspect 11 is a sensor device according to one of the preceding aspects, wherein the sensor element further comprises a further resistor element arranged in the cavity, a sealed reference cavity and two reference resistor elements arranged in the reference cavity, wherein the resistor element, the further resistor element and the two reference resistor elements are arranged in a Wheatstone bridge, and wherein the power supply unit is further configured to apply the voltage to the Wheatstone bridge.

Aspect 12 is a sensor device according to Aspect 11, wherein a respective one of the resistor elements and one of the reference resistor elements form a voltage divider of the Wheatstone bridge.

Aspect 13 is a sensor device according to one of the preceding aspects, wherein the evaluation unit for determining the gas concentration is configured to compensate the first output signal using the second output signal.

Aspect 14 is a sensor device according to one of the preceding aspects, wherein the evaluation unit for determining the gas concentration is configured to apply a filter operation to the first and/or the second output signal, in particular a Kalman filter operation or a sliding average operation.

Aspect 15 is a sensor device according to one of the preceding aspects, wherein the evaluation unit for determining the gas concentration is configured to apply a correction factor to the first and/or the second output signal, in particular a temperature-dependent correction factor.

Aspect 16 is a sensor device according to one of the preceding aspects, wherein the control unit is configured to detect a multiplicity of first output signals at the applied first voltage, and wherein the evaluation unit is configured to determine the gas concentration based on the multiplicity of the first output signals and the second output signal.

Aspect 17 is a sensor device according to one of the preceding aspects, wherein the control unit is configured to set the first voltage and the second voltage such that the first output signal essentially depends on an ambient pressure of the gas and the second output signal essentially depends on the thermal conductivity of the gas.

Aspect 18 is a sensor device according to one of the preceding aspects, wherein the gas concentration is a hydrogen concentration.

Aspect 19 is a sensor device according to one of the preceding aspects, wherein the sensor element is a thermal conductivity sensor.

Aspect 20 is a method for determining a gas concentration comprising:
surrounding a resistor element of a sensor element with a gas, applying a first voltage to the sensor element, detecting a first output signal of the sensor element with the first voltage applied, applying a second voltage to the sensor element, which is different from the first voltage, detecting a second output signal of the sensor element with the second voltage applied, and determining the gas concentration based on the first output signal and the second output signal.

1. A sensor device for measuring a gas concentration, comprising:
a sensor element comprising a cavity with an opening for receiving a gas, and a resistor element arranged in the cavity;
a power supply unit configured to apply a voltage to the sensor element;
a control circuit configured to:
set a first voltage of the power supply unit and detect a first output signal of the sensor element while the first voltage is applied to the sensor element, and
set a second voltage of the power supply unit and detect a second output signal of the sensor element while the second voltage is applied to the sensor element, wherein the second voltage is different from the first voltage;
an evaluation circuit configured to determine the gas concentration based on the first output signal and the second output signal.

2. The sensor device as claimed in claim 1, wherein at least one of the first output signal or the second output signal depends on the gas concentration and a thermal conductivity of the gas.

3. The sensor device as claimed in claim 1, wherein the control circuit is configured to:
cause a temperature of the resistor element to reach a first temperature using the first voltage and determine the first output signal at the first temperature, and
cause the temperature of the resistor element to reach a second temperature using the second voltage and determine the second output signal at the second temperature,
wherein the first temperature is different from the second temperature.

4. The sensor device as claimed in claim 3, wherein at least one of the first temperature or the second temperature is greater than an ambient temperature of the sensor device.

5. The sensor device as claimed in claim 3, wherein at least one of the first temperature or the second temperature depends on the gas concentration and a thermal conductivity of the gas.

6. The sensor device as claimed in claim 1, wherein the control circuit is configured to determine a first resistance signal of the sensor element as the first output signal and a second resistance signal of the sensor element as the second output signal.

7. The sensor device as claimed in claim 1, wherein the resistor element is in the form of a micro-electromechanical systems (MEMS) structure.

8. The sensor device as claimed in claim 1, wherein the resistor element has piezoresistive properties.

9. The sensor device as claimed in claim 1,
wherein the sensor element further comprises a sealed reference cavity and a reference resistor element arranged in the sealed reference cavity, and
wherein the power supply unit is further configured to apply the voltage to the resistor element and to the reference resistor element of the sensor element.

10. The sensor device as claimed in claim 9, wherein the resistor element and the reference resistor element are arranged in a parallel switching configuration.

11. The sensor device as claimed in claim 1,
wherein the sensor element further comprises a further resistor element arranged in the cavity, a sealed reference cavity, and two reference resistor elements arranged in the sealed reference cavity,
wherein the resistor element, the further resistor element, and the two reference resistor elements are arranged in a Wheatstone bridge, and
wherein the power supply unit is further configured to apply the voltage to the Wheatstone bridge.

12. The sensor device as claimed in claim 11, wherein a respective one of resistor element or the further resistor element and one of the two reference resistor elements form a voltage divider of the Wheatstone bridge.

13. The sensor device as claimed in claim 1, wherein the evaluation circuit is configured to compensate the first output signal using the second output signal.

14. The sensor device as claimed in claim 1, wherein the evaluation circuit configured to apply a filter operation to at least one of the first output signal or the second output signal.

15. The sensor device as claimed in claim 1, wherein the evaluation circuit is configured to apply a correction factor to at least one of the first output signal or the second output signal.

16. The sensor device as claimed in claim 1,
wherein the control circuit is configured to detect a multiplicity of first output signals at the first voltage, and
wherein the evaluation circuit is configured to determine the gas concentration based on the multiplicity of the first output signals and the second output signal.

17. The sensor device as claimed in claim 1, wherein the control circuit is configured to set the first voltage and the second voltage such that the first output signal depends on an ambient pressure of the gas and the second output signal depends on the thermal conductivity of the gas.

18. The sensor device as claimed in claim 1, wherein the gas concentration is a hydrogen concentration.

19. The sensor device as claimed in claim 1, wherein the sensor element is a thermal conductivity sensor.

20. A method for determining a gas concentration, comprising:
surrounding a resistor element of a sensor element with a gas;
applying a first voltage to the sensor element;
detecting a first output signal of the sensor element with the first voltage applied;
applying a second voltage to the sensor element, which is different from the first voltage;
detecting a second output signal of the sensor element with the second voltage applied; and
determining the gas concentration based on the first output signal and the second output signal.
